(19) United States
(12) Patent Application Publication
(10) Pub. No.: US 2023/0081359 A1
(43) Pub. Date: Mar. 16, 2023

(54) Digital mental health ecosystem

(71) Applicants: Seyedeh Leili Mirtaheri, Rende (IT); Reza Shahbazian, Rende (IT); Ali Eslami, Vancouver (CA)

(72) Inventors: Seyedeh Leili Mirtaheri, Rende (IT); Reza Shahbazian, Rende (IT); Ali Eslami, Vancouver (CA)

(21) Appl. No.: 17/994,308

(22) Filed: Nov. 26, 2022

(51) Int. Cl. G16H 50/20; G16H 10/60

(52) U.S. Cl. CPC G16H 50/20; G16H 10/60

(57) ABSTRACT

A digital mental health ecosystem is presented to address all the mental health disorders and improving the treatment process patients need. Human behavior and physiological factors are studied and proper solution for each individual is presented to caregivers and their respective patients.

### BACKGROUND OF THE INVENTION

It is stated that in the modern era, nearly 1 billion people live with a mental disorder and, particularly in low-income countries, more than 75% of people with various mental disorders do not receive any form of treatment. Considering all the people worldwide who are dealing with mental problems, about 50% of mental health disorders are claimed to start by the age of 14. In addition, close to 3 million people around the world die annually due to substance abuse, and every 40 seconds, a person dies by committing suicide.

It is estimated that over 160 million people need humanitarian assistance because of personal or social conflicts, natural disasters, or other emergencies. The rates of mental disorders can double during such crises. Furthermore, one in five people affected by such conflicts is estimated to have a mental health condition.

Therefore, understanding human behavior has become one of the most promising areas in the realm of healthcare research. In this regard, many researchers have shown that following a healthy lifestyle can promote health, prevent disease and slow its progression. While unhealthy lifestyles and deleterious habits such as sedentary lifestyles, poor diets, anxiety, and sleep disorders can greatly increase the risk of disease and premature death.

The important point is that the identification of the human behavior process is complex and depends on many physical, psychological, and social factors. Accurate recognition of human behavior has still remained a major challenge and has attracted a great deal of research effort. This identification can suggest a mentally and psychologically healthy lifestyle to people according to their moods and behaviors and, consequently, greatly reduce the duration of the treatment process. The treatment process is also done traditionally with not enough information using patient's behavior and effective elements on their illness. The following patent application proposes solutions to overcome the aforementioned shortcomings.

### BRIEF DESCRIPTION OF THE DRAWINGS

FIG. 1, Displays the architecture of the proposed model

FIG. 2, displays the use case diagram of the proposed system (101)

FIG. 3, displays the comprehensive flowchart (201) of the proposed system

FIG. 4, displays the digital intake management flowchart designed for patients (102-1)

FIG. 5, displays the digital intake management flowchart designed for supporters (102-2)

FIG. 6, displays the digital symptom management designed for patients (001) (105-1)

FIG. 7, displays the digital symptom management designed for supporters (105-2)

FIG. 8, displays the digital crisis management designed for patients (113-1)

FIG. 9, displays the digital crisis management designed for supporters (113-2)

FIG. 10, displays the digital diagnose management (103) of the proposed system

FIG. 11, displays the screening management (104) of the proposed system

FIG. 12, displays the intervention/treatment management (107) of the proposed system

FIG. 13, displays the assigning counselor (003) procedure (112)

FIG. 14, displays the digital behavior management (108) of the proposed system

FIG. 15, displays the report management (111) procedure of the proposed system

FIG. 16, displays the social network related process designed for counselors (168-3)

FIG. 17, displays the social network process considered for patients (168-1)

FIG. 18, displays the social network procedure designed for supporters (168-2)

### DEFINITIONS

The following are the definition of each one the described layers and steps of the preferred embodiments.

| | |
|---|---|
| Patient | 001 |
| Support | 002 |
| Counselor | 003 |
| Social | 004 |
| Usecase Diagram | 101 |
| Digital intake Management | 102 |
| Diagnose Management | 103 |
| Digital screening Management | 104 |
| Digital symptom Management | 105 |
| Clinical Supervision | 106 |
| Digital intervention/Treatment Management | 107 |
| Digital Behavior Management | 108 |
| Detecting Inconsistency | 109 |
| Alert Management | 110 |
| Report Management | 111 |
| Assignation a counselor | 112 |
| Digital crisis Management | 113 |
| Selecting standard Questionnaires for patients | 114 |
| Selecting standard Questionnaires for caregivers/supporters | 115 |
| Designing and creating, Editing Questionnaires for patient | 116 |
| Designing and creating, Editing Questionnaires for | 117 |
| caregivers/supporters | |
| Designing and creating the flow of Questionnaires | 118 |
| Data Analysis | 119 |
| Symptom Detection | 120 |
| Symptom Selection | 121 |
| Symptom Tracking | 122 |
| Symptom Monitoring | 123 |
| Identify Feel Better Tools | 124 |
| Identify Early Warning Signs | 125 |
| Identify Stressors | 126 |
| Linking to Supporter App | 127 |
| Add Supporter and Trusted People contact numbers | 128 |
| Add Safe Location | 129 |
| Location Tracking | 130 |
| Check in | 131 |
| Check in Analysis | 132 |
| Monitoring the locations based on the safe/not safe | 133 |
| Urgent Call request by patient | 134 |
| Suggest to call to Trusted People | 135 |
| Suggest to do feel better tools | 136 |
| Unsafe Locations from Supporter Safety App | 137 |
| Identify Feel Better Tools for patient | 138 |
| Identify Early Warning Signs for patient | 139 |
| Identify Stressors of patient for patient | 140 |
| Check in about patient | 141 |
| Propose Social Network | 142 |
| Propose the Guideline | 143 |
| Add Safe Location | 144 |
| Add Unsafe Location | 145 |
| Compare the safe locations | 146 |
| Monitoring the location of the patient based on | 147 |
| the safe/not safe | |
| Detecting Common Locations | 148 |
| Receive notifications | 149 |
| Request Status Update | 150 |
| Receive Child Status is Updated | 151 |
| Patient Risk Profile | 152 |
| Clinical Diagnosis | 153 |
| Automatic Diagnosis | 154 |
| Clinical based Intervention/Treatment | 155 |
| Technological based Intervention/Treatment | 156 |
| Report Generation | 157 |
| Psychotherapeutic mindfulness and ACT | 158 |
| Psychotherapeutic interventions | 159 |
| Gamified interventions | 160 |
| Preventive interventions | 161 |
| Psychotherapeutic CBT | 162 |
| Measurement and follow up | 163 |
| Supportive interventions | 164 |
| Skills training | 165 |
| Behavioral intervention | 166 |
| Chatbot | 167 |
| Social Media | 168 |
| Applications | 169 |
| Smart Clothes | 170 |
| Extended reality | 171 |
| Selecting Doctor/Clinician/ Counselor by Patient | 172 |
| Selecting Doctor/Clinician/ Counselor by Supporter | 173 |
| View other people's comments about the Counselor | 174 |
| Payment Management | 175 |
| Linking Counselor to patient App | 176 |
| Making Appointment | 177 |
| Online meeting | 178 |
| Chatting and Calling to Counselor | 179 |
| Scoring to the Counselor | 180 |
| Putting Comments about the Counselor | 181 |
| Changing the Counselor | 182 |
| Gathering Passive Data by mobile/digital device | 183 |
| sensor tracking | |
| Gathering Active Data by interacting with patient and | 184 |
| supporters | |
| Data Acquisition | 185 |
| Data Cleaning | 186 |
| Interpret the results | 187 |
| System or human scribing -drafting the report and | 188 |
| tagging the video and audio in the report | |
| Preparing the clinical report by human | 189 |
| System Report Engine | 190 |
| Social network Management | 191 |
| Intake report | 192 |
| Digital Behavior report | 193 |
| Screening report | 194 |
| Intervention/Treatment report | 195 |
| Counselor prepared report | 196 |
| Symptom management | 197 |
| Social network report | 198 |
| Create Public Profile | 199 |
| like/dislike comments | 200 |
| Put comments about posts | 201 |
| View the recommended post based on his/her favorites and | 202 |
| similarity | |
| View the recommended users by common favorites | 203 |
| View the list of club houses and in which she/he is joined | 204 |
| Request to Join to the club houses of Counselor | 205 |
| Create/edit/delete/post the therapeutic Content to publish in | 206 |
| the explore | |
| Join to the Club house of the Counselor | 207 |
| Receive a not permission message | 208 |
| Inserting/deleting/editing text, voice | 209 |
| Leave it | 210 |
| Edit/Delete Public Profile | 211 |
| Share/Un-sharing the Favorite music, photos, videos, | 212 |
| Daily and Check-in in public profile | |
| Sharing/Un-sharing the Favorite music, photos, videos, s | 213 |
| Daily and Check-in to Follower | |
| Send/Edit/Delete Posted music, photos, videos, Daily and | 214 |
| Check-in to Explore | |
| Send/Edit/Delete Post music, photos, videos, Daily and | 215 |
| Check-in to a Follower | |
| Follow/Unfollow | 216 |
| Content Management analysis | 217 |
| Creating Profile | 218 |
| View the list of club houses and in which she/he is joined | 219 |
| Join to club houses of Counselor | 220 |
| Creating the Subject-oriented rooms | 221 |
| Joining the Subject-oriented rooms by other supporters | 222 |
| Edit/Delete Profile | 223 |
| Inserting/editing/deleting text, voice, photos | 224 |
| view the score of the comments and users | 225 |
| Create/edit/delete club houses and view the list of | 226 |
| club house | |
| Join to club houses of other counselors | 227 |
| Private chat with patient/supporter | 228 |
| Fog Layer | 1100 |
| Fog node | 1110 |
| Cloud Layer | 1200 |
| Cloud center | 1210 |
| Data Analysis Layer | 1300 |
| Data Acquisition | 1310 |
| Data Cleaning | 1320 |
| Data Analysis | 1330 |
| Digital Health Layer | 1400 |
| People interaction Layer | 1500 |

### DETAILED DESCRIPTION OF THE PREFERRED EMBODIMENT

Mental health disorders are widely considered a major problem that has strongly influenced the well-being of people. Moreover, tackling this issue promptly has become even more crucial, taking into account the absence of proper treatments and lack of access to immediate care in many countries, in addition to the decreasing trend of age associated with individuals diagnosed with mental problems. In this regard, researchers have proposed the notion of investigating one's mood change as a promising approach to assist them to promote their well-being and have a healthy lifestyle, consequently, reducing the risk of developing mental health disorders and improving the treatment process patients need to undergo. However, perceiving human behavior has remained challenging due to its unique context, in addition to the necessity of considering various social and psychological factors while studying one's mood change. As a result, the following patent application is proposed to tackle the common shortcomings of the traditional existing approaches and presented a proper solution considering different aspects of one's treatment process and tracking the trend of behavior change.

### Intake Management:

Intake management is the first step of providing the care and treatment service when a patient (001) seeks help for their mental health issues. It mainly includes checklists of considerations as health questionnaires, which helps counselors (003) better understand their patients (001) to determine the appropriate treatment options. In addition, it helps psychologists to support their well-being in the long term by providing prominent background information.

A more detailed look will indicate that an intake management for therapy is a specialized comprehensive document that uncovers the root causes of mental illnesses and reveals formerly not seen psychological and physical issues that can be used to inform the treatment plan being made for the patients (001) in clinics and hospitals. Specifically, an intake management made for mental health issues gives crucial details on a patient's present mental state, their risk of deleterious actions such as self-harm and suicide, and the appropriate and requisite treatment and medications needed to help healthcare providers deliver the most qualified care.

The questionnaires utilized during an intake management generally include various types of questions about different aspects of a patient's life, including family related issues, personal demographic details, current medical, psychological and emotional state and present problems that patients (001) might face in their lives and safety plans and crucial needs and supports for the time of crisis.

The information gained through the intake management process provides both patients (001) under treatment and counselors (003) a scheme of the state in which the patient (001) is present and the state they want to reach at the end of the therapy process, along with highlighting the most concerning issues either from the patient's and the psychologist's perspective. Having this information been provided, long-term treatment plans can be developed more straightforwardly, prioritizing crucial problems. Treatment plans are the courses of actions recommended by the counselors to be taken so as to treat the patient (001) to deal with their mental health issues. In this case, some concealed issues behind those superficial problems can be uncovered which tackling them in the first place will prominently push forward the process of treatment in a way that both the help seeker and the service provider benefit from. For instance, the best possible course of treatment can be determined based on the details provided during the intake management in addition to finer understanding of the obstacles and the core problems patients (001) are struggling with particularly for long-term.

Regarding the suicidal patients, in particular, their assessment is one of the most demanding and stressful tasks. Therefore, a proper and professional intake management process can be regarded as a systematic model to obtain suicide-relevant information and thoughts that patients (001) are often reluctant to openly discuss. This can help the psychologists to detect the most prominent suicide risk factors present in a patient's daily life or behavior, and to determine if the intake process should consider greater focus on suicide assessment and its symptoms like depression, suicide ideation and suicide attempts. That is to say, intake management critically needs precise and acute observation to identify any little behavior which might be associated to suicidal thoughts. Furthermore, in some cases, especially when it comes to a patient (001) who can be found of harboring suicidal thoughts, questioning calmly yet directly about patient's emotions and thoughts will become necessary.

Evaluating Suicide-related intention during an intake management process is another vital area that can be established by various types of reports or behavioral observations during interviews. As a result, questionnaires can be used in conjunction with personal interviews during an intake management to obtain more related information.

### Symptom Management:

Symptom management (105) is one of the essential steps counselors (003) take in preparing mental health treatment plans for patients (001) suffering from mental illnesses and health issues. Noticeably, the process associated with the symptom management can be regarded as one of the important phases of mental health treatment. This can not only help patients (001) to understand the effects of their problems in their daily life but also assists the counselor (003) in tracking the patient's changes in behavior, emotion and action to adjust the treatment developed for the patient (001) properly. However, the symptom management process cannot be understood as a viable goal for mental health treatments due to being a disservice to patients (001) and the aim of health care services.

A symptom management (105) process comprises identifying the symptoms of an illness, side effects caused by the treatment considered for a patient (001) having either mental or physical problems, and psychological, social and emotional problems a patient (001) might encounter while suffering from a disease. The outcome of the process mentioned above is the symptoms and symptoms status that a patient (001) indicates in behavior, action, and emotion.

Symptoms are subjective to each patient, and even in two patients (001) with the same mental issue, their symptoms are not necessarily alike. Even if their symptoms were the same, the severity of the symptoms would show how profound their problem is. In addition, in many cases, there is no agreement as a standard measurement on the amount of time symptoms will persist and the time symptoms can be considered as signs for progression or recurrence of the health issue. Symptoms can significantly affect a patient's life in various ways, especially when residual symptoms attenuate a patient's quality of life by issuing physical and emotional functioning problems.

It should be noted that symptom management (105) in the presented model should not be considered just a process that patients (001) go through in order to control and alleviate their pain and other intrusive symptoms as is regarded in other medical models, especially when a patient (001) is in imminent risk of death or suffering from painful medical conditions, particularly during their post-treatment period. With the later definition of this process, mental health treatment plans might seem not leading to real healing and improvement, whereas with the symptom management (105) employed here, symptoms are tracked in order to develop the best possible treatment plan and revise it as the treatment shows its effect on patients (001) and their symptoms.

### Crisis Management:

Generally, a crisis is a deleterious event that threatens to harm an organization, a group of people or individuals. Particularly, in the case of mental health, a crisis can be seen as not necessarily a traumatic event or emergence of a mental condition that may cause severe and intolerable pain or difficulty for a patient (001) but as a person's reaction to an event that differs among individuals according to their characters and their tolerance when facing the same event. According to Caplan's definition in 1961 and 1964, a crisis happens when a person faces an “insurmountable obstacle by the use of customary methods of problem-solving”. Such occurrences can result in feeling overwhelmed, helpless, Anxious, hopeless and in more severe cases it may cause serious panic attacks.

However, it can be argued that crisis events happen abruptly, and no one can precisely estimate when and how a harsh, threatening event might happen, especially in the case of mental health when a patient (001) might act healthful at a time and then suffer from a severe psychological and mental issue, they are somehow expectable and can be caused by a range of reasons.

Crisis management can be defined as a situation-based and systematic attempt to prevent potential crises or help manage situations when some crisis occurs. Despite the differences in crisis definitions, being surprising and a necessity for making decisions immediately and in a short time are two common elements to this concept. Thus, considering the need of expediting solutions when a crisis occurs highlights the importance of having premade plans and being prepared for such a situation by covering every eventuality, the worst-case scenarios while suggesting a number of possible solutions for each potential case. Such plans are usually referred to as crisis management plans (or safety plans), including actions which could be done when a patient is expected to experience a crisis in order to promote their well-being and preclude them from taking any self-harm actions. While making a crisis management plan, scenarios which are considered crises and should trigger the necessary and suitable response mechanisms to the situation in hand are defined and relevant metrics to this definition are established. In addition, apart from including all the roles and responsibilities, the crisis management plan should also consider proper action in different states of crisis prevention and assessment, handling the crisis and agreeing on the termination rules.

In the crisis management, both obvious events (such as losing a job, experiencing an accident or the death of someone close) and less apparent one that can be known as a crisis should be considered. In later cases, a person may not openly show related feelings of being in a state of crisis, while the experienced event can still cause drastic changes in behavior and mood, which should not be ignored. Such changes may include physical changes like losing or gaining weight, sleep disturbance, or psychological changes such as decreased performance at work or school, neglect of social communication and hygiene and abrupt and dramatic mood changes.

One should grasp the importance of providing accurate information while preparing a crisis management plan. Provided that the information is accurate and the plan indicates proper steps and how quickly actions should be performed, one should be able to handle the situation quickly and efficiently. Also, the required information is expected to cover possible consequences in both the short and long term. On the other hand, incorrect or manipulated information may not help decision makers successfully pass the crisis but can also exacerbate the situation.

### Diagnose Management:

Diagnosis is one of the steps taken to identify the leading causes of a problem a patient (001) is suffering, along with history taking and examination. An accurate and appropriate diagnosis can help patients (001) rapidly return to their healthful state by offering a straightforward treatment plan and providing valuable information about their health condition.

As a result, clinical diagnosis is the process of determining whether the symptoms and behavioral patterns that a patient (001) presents match the diagnostic criteria of a mental illness, using the data provided during assessments and performing within a diagnostic framework, and mainly according to the established classification systems such as DSM-5 (The Diagnostic and Statistical Manual of Mental Disorders) and ICD-10 (International Classification of Diseases 10th Revision).

Specifically, in the case of mental health, psychological diagnostic testing is a process comprising standard psychological examinations by which psychologists can diagnose mental illnesses. These psychological tests assess a patient's several factors including behavior, mental ability and cognition while predicting possible future outcomes of the patient's mental health. In addition, psychological diagnostic tests are also used to assess a person's intelligence and neuropsychological functioning and determine whether a specific treatment plan is suitable for a patient. Noteworthy, the diagnostic tests can be taken several times and regularly during a patient's treatment to develop the treatment plan, making sure that the plan is according to the recent diagnosis of the patient (001) and the symptoms indicated by them. This can also happen when a patient (001) is not satisfied with the diagnosis and the treatment considered for the patient.

Due to the fact that psychologists use the tests mentioned above to collect required information about a patient's mental health status, such tests are part of the psychological assessment process that measures specific characteristics. In this regard, a psychologist can use cognitive and personality tests that help professionals diagnose psychiatric disorders and become familiar with the patient's unique thoughts, behavior, and psychological state.

Generally, reaching a diagnosis may need different types of tests, such as a physical exam to determine physical problems causing the symptoms, some laboratory tests and a psychological evaluation in which a mental health professional asks questions about the patient's symptoms, thoughts and feelings and behavior patterns and might need the patient (001) to fill some questionnaires. By differentiating the physical and psychological causes of mental illness, along with considering the circumstances that the problem occurs, professionals are able to develop an effective plan to treat a mental disorder. Notably, diagnosing a mental illness accurately and finding out the real causes of symptoms needs minute attention, effort and spending time.

### Screening Management:

Screening management is a process that can help counselors (003) identify one or more particular diseases at an early stage. It involves medical tests that can be used for asymptomatic patients (001) who might not present any specific sign of disease but be at risk. As a result, screening tests are usually performed on participants who do not have a clinical indication of diseases. Tracking patients (001) in a screening management and regular follow-ups can provide the effective information to treat patients (001) and the therapeutic benefits of detecting diseases earlier, enabling in time intervention and management to reduce mortality and suffering. This process—along with the assessment—initially occurs at the intake step and should continue throughout the treatment course to promptly determine any change in the symptoms and the effect of the treatment on the patient. Such routine tracking of a patient's mental health helps the professionals to screen how the treatment is going on. Consequently, the counselor's (003) comprehension of the patient's behavioral patterns, symptoms and problems strongly influence the type of interventions and how long a treatment should be applied.

Screening management has been used in several areas such as health tracking in patients (001) suffering from cancer, drug usage, various diseases, and screening medical health issues and problems in students and employees in their workplace. However, despite leading to an early diagnosis of the potential problem a patient (001) might suffer, one should be aware of the effect of over-diagnosis or misdiagnosis that could have some possible deleterious effects of the patient's treatment. Thus, a proper screening management needs to have high sensitivity and specificity. Furthermore, before a screening test is taken into practice, it needs to be thoroughly checked to make sure that it will not do any potential harm and ensure that the results will not be influenced positively owing to the biases in the study method. Although this inherited bias can affect the results in a misleading way, it can be precluded by utilizing a randomized controlled trial.

As a whole, even though there are some potential problems related to screening, it is still a beneficial approach for increasing the health of patients (001) with concealed medical issues and preventing more serious diseases in the future. In addition, guidelines have been published by WHO in 1968 to improve this process and reduce its possible disadvantages. These guidelines take several conditions into account like: the screening management should be done for a vital health problem with a possible treatment and with available diagnosis and treatment facilities.

Screening management is typically conducted in brief interviews and/or self-reports questionnaires or report questionnaires filled by a patient's supporters (002). Also, the instruments for the screening management, aim to assess any disturbance or developmental backwards in patients' treatments. These are mainly used as primary steps to detect a particular medical issue in advance and to identify a more comprehensive assessment can be done when it is needed. This can help professionals to perceive if any inclusion in a treatment plan is needed. As a result, the treatment developed for the patient (001) might be expected to have higher possibility of success due to the early diagnosis and less severity of disease.

### Intervention and Treatment Management:

Interventions used for psychological issues often follow some standard assessments to identify all the relevant aspects of emotional reactions to different problematic occurrences such as drastic and sudden change, the loss of someone close, or trauma. Psychological interventions are used for various purposes from a more specialized to general comprehensive ones and are claimed to be effective not only in improving pain, psychological issues but also in helping patients (001) to improve their physical activity.

During the intervention and treatment management, an appropriate treatment plan will be made according to the patient's present situation and the developed treatment plan is checked to make sure than the treatment is going on based on the proper plan considered for promoting the patient's well-being and mental health. In order to do so, every nuance of change in symptoms or patient's behavior patterns should be taken into account. As a result of tracking the patient's response to the treatment, any potential harm or misdiagnosis can be found in the early stage, preventing the patient (001) from suffering serious and severe illness or side effects.

The advancement of technology has provided opportunities to enhance the potency of psychological interventions by using digital technologies. Broad access to mental health care, helping patients (001) to self-manage their medical conditions, expanding therapeutic approaches to daily life and increasing the exposure of psychological intervention are of the benefits gained from utilizing digital tools. In addition, mental health professionals are able to access more people who are seeking help with the potential of developing self-guided interventions, beyond the possible number of people in face-to-face therapeutic interviews.

### DETAILED DESCRIPTION OF THE INVENTION

### Proposed Architecture

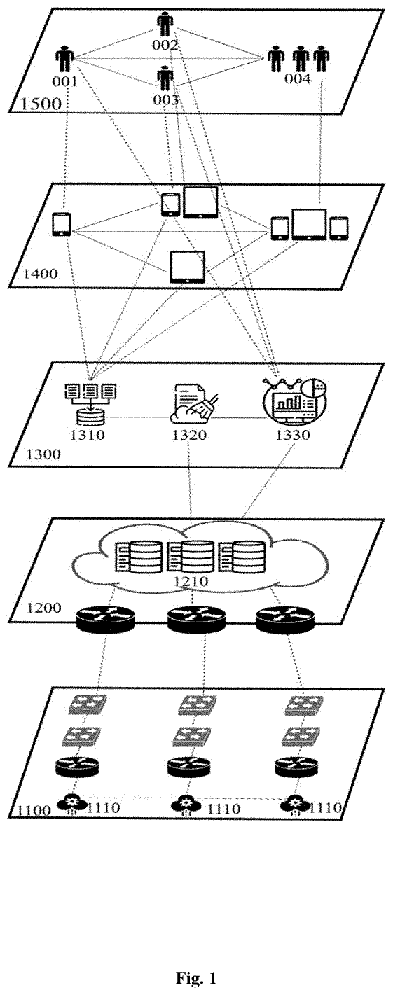

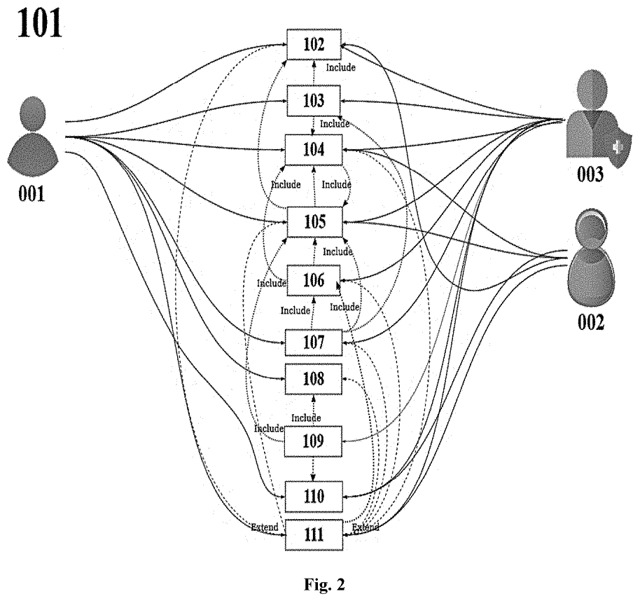

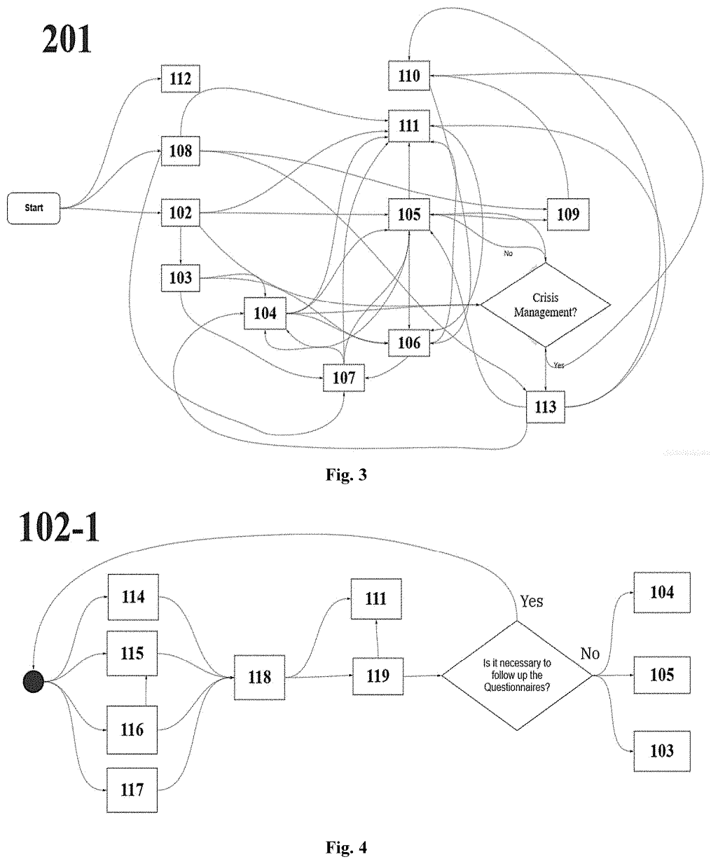

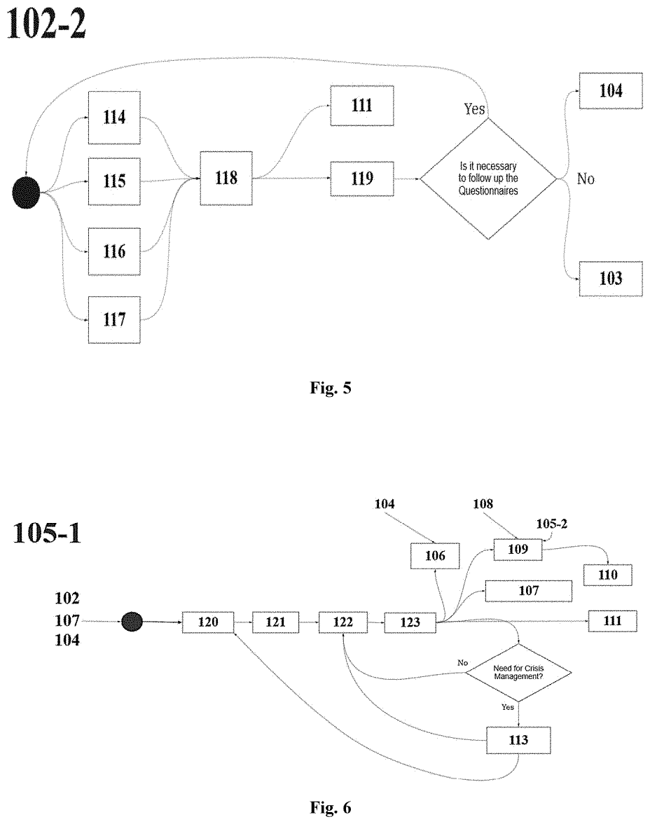

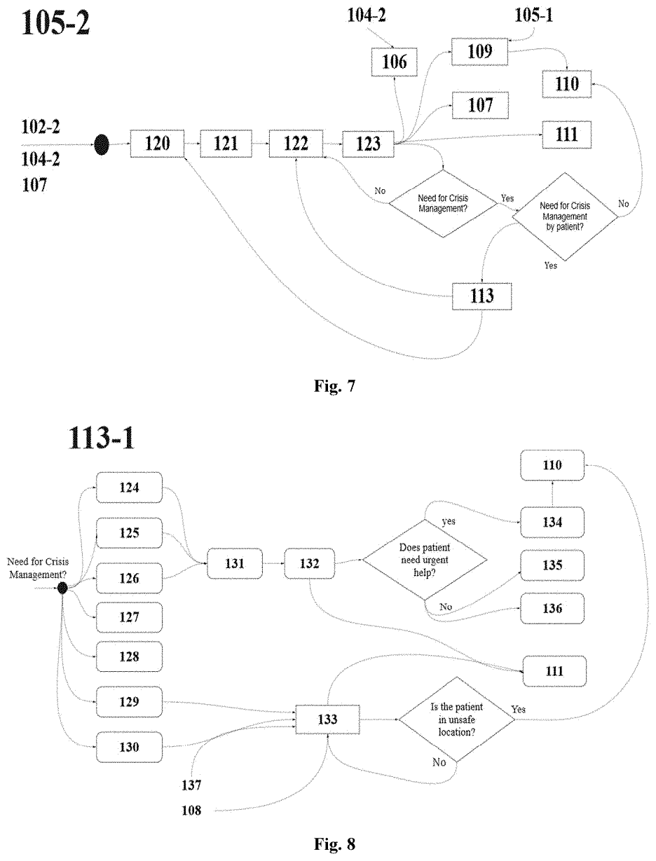

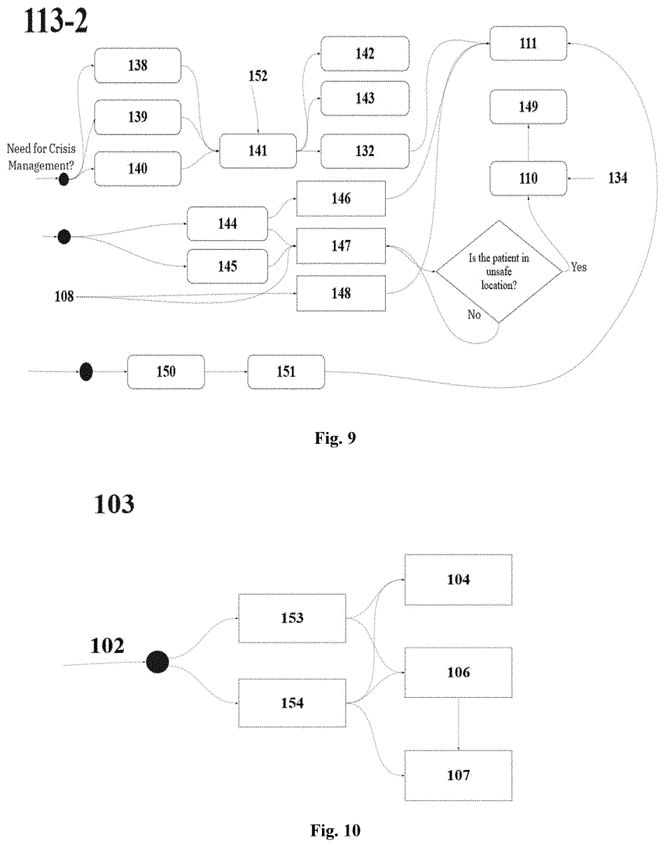

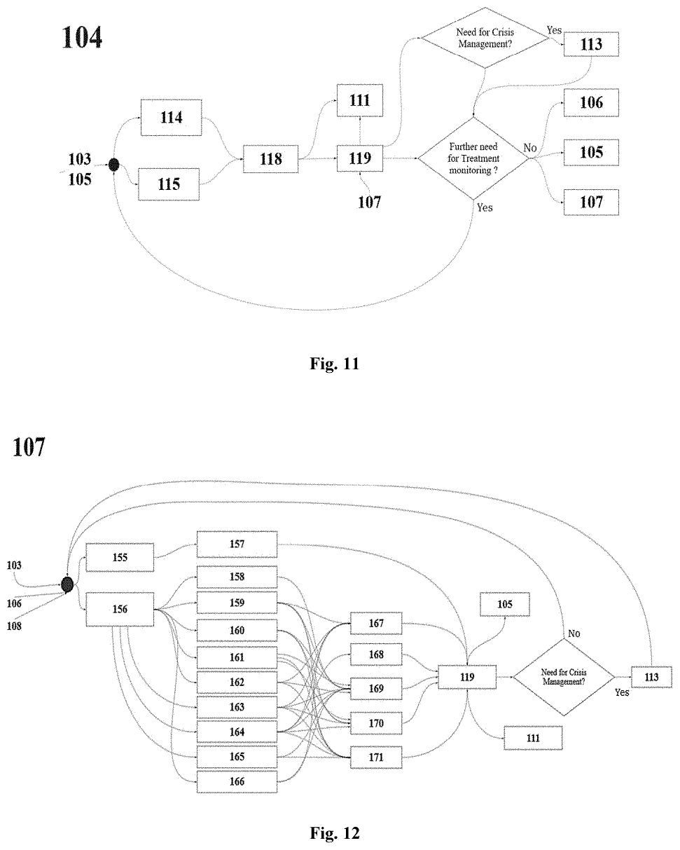

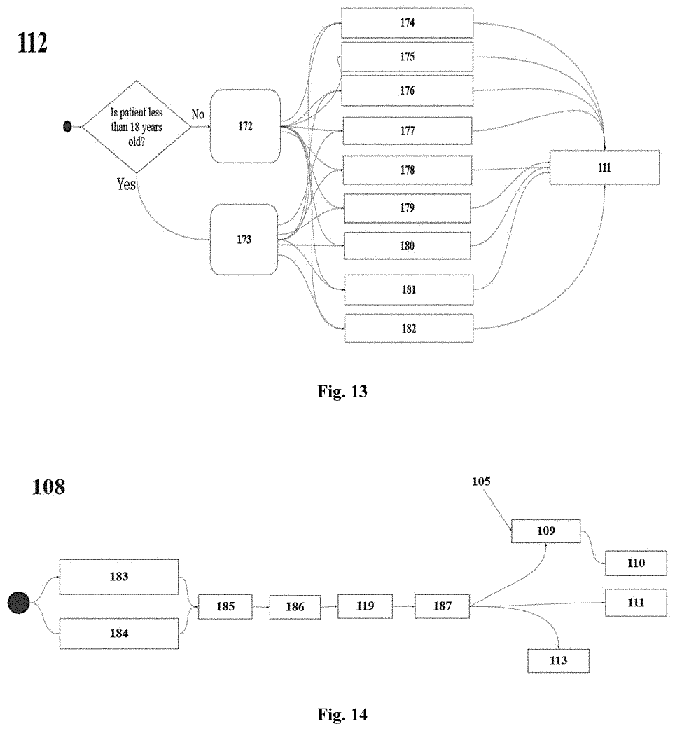

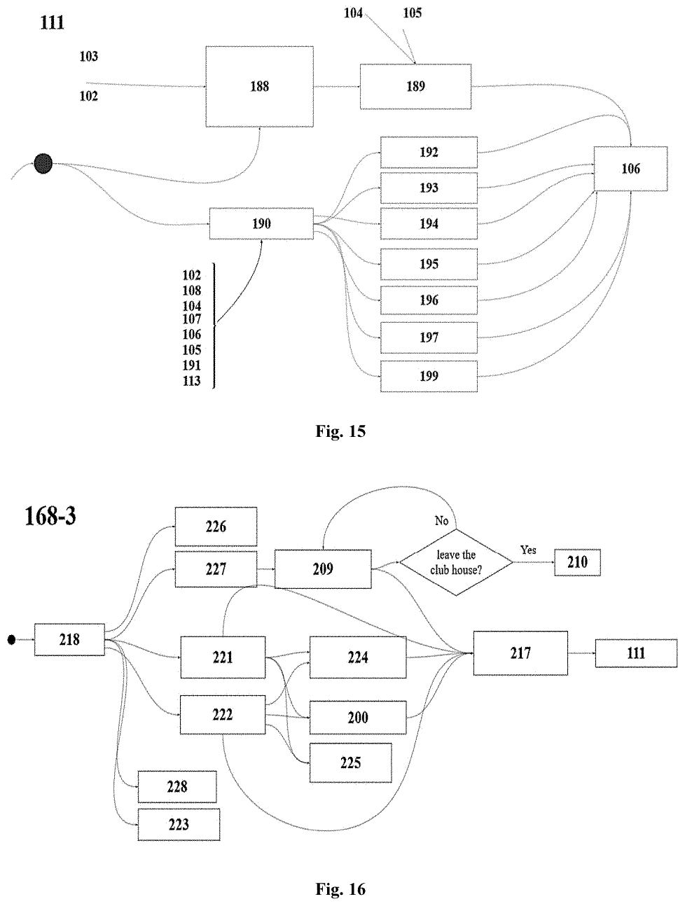

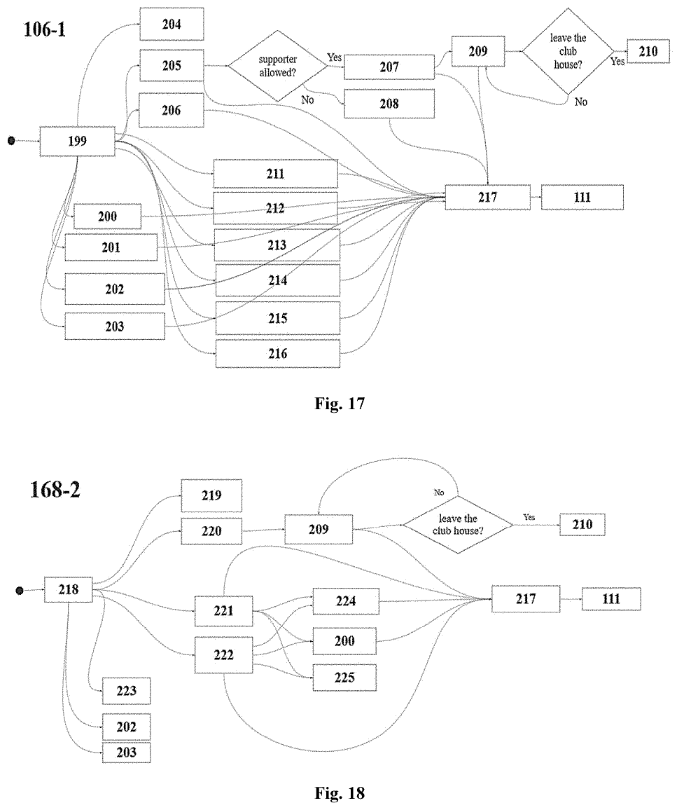

To handle the digital mental health process efficiently, there ought to be an architecture to help different aspects of treating a mental issue interact with each other and play their own roles. The following sections will describe the architecture of the proposed model in a succinct way yet thoroughly. The general view of the model's architecture will illustrate that the presented model comprises of five distinct layers (See FIG. 1) according to each aspect of dealing with treating people who are seeking help for mental illnesses, in particular suicidal ideations. The main layers of the model include the followings (the layers are mentioned in a bottom-top order):
1) Fog layer 1100: Handling the computational aspect
2) Cloud layer 1200: The backbone for data storage and providing real-time services
3) Data analysis layer 1300: Data acquisition, cleaning and analysis
4) Digital health layer 1400: A group of interactive connected applications to help patients (001) in dealing with their mental issues and tracking their mood and behavior
5) People interaction layer: The interactive connection among patients (001), supporters (002), counselor (003) and social community (004).

### Fog Layer (1100)

Due to the fact that there is a need for instant decision making as well as data security matters, regarding the gathering of extensive information from so many sources, analyzing them and providing real-time reactions and answers, the need for a fog layer in addition to the cloud layer, in this architecture seems so apparent. As a result, there is a fog layer (1100) behind a cloud layer (1200) provided in the proposed models' architecture which generally is used for computational matters.

The fog layer (1100) comprises of several fog nodes (1110) which are responsible for the computational aspect of the problem. These nodes (1110) can essentially be regarded as elements that provide computational power as well as storage and connectivity service, and include devices like I/O devices, switches and gateway computers and will extend the cloud layer (1200) to be closer to the devices of the network. Unlike the cloud layer (1200) that focuses on data processing and secure data storage, the fog layer (1100) concerns about providing moving computational power, device controlled capacity and networking power close to the devices and providing lighter real-time services such as location tracking.

### Cloud Layer (1200)

To satisfy concerns regarding the data security and centralized data processing, there ought to be a cloud layer (1200) in the model's architecture. The cloud layer (1200) helps in storing data in a secure manner owing to the potential alteration or deletion of the data that can be accessed by the service provider at any time, as well as providing real-time service and answers for the users.

The cloud layer (1200) comprises of several cloud servers (1210) to store the data gathered from multiple resources and analyzed in the data analyzing layer, and provides agile, flexible, and device and location independent service. Also, the heavier services that need more computational power and resources will be done in the cloud layer (1200).

### Data Analysis Layer (1300)

This layer focuses on gathering required data from various sources of information in its upper layer, meaning the Digital Health layer (1400). This layer (1300) comprises of three main sections: data acquisition (1310), data cleaning (1320) and data analysis (1330). In the data acquisition section, required data and information about the mental health condition of patients (001) will be gathered (1310) from various smartphone applications and other relevant IOT devices they interact with as a form of passive sensing of their behavioral patterns and mood changes.

Next, this gathered data will be cleaned (1320) by removing any possible noise and to provide useful data to be analyzed (1330) in the next step. Data cleaning process (1320) is important due to the fact that there is a high chance of duplicated or mislabeled data while combining data from multiple resources. Such incorrect information will lead to incorrect outcomes and unreliable results. Although the data cleaning process (1320) varies from application to application and is dependent on the employed dataset, it usually includes fixing or eliminating incorrect, corrupted, incomplete data and duplicates and incorrectly formatted data.

Finally, data will be analyzed (1330) using various yet appropriate tools to provide results indicating the changes in the patient's mood and behavioral patterns, the frequency of suicidal thoughts and ideation and the risk of suicide in the near future. The results gained by analyzing the gathered and cleaned data will be formed as comprehensive reports and will be sent to the patients (001), supporters (002), and counselors (003) to help them track the improvements made in treating the patient. These reports can assist the counselor (003) to examine the effectiveness of the treatment plan developed for the patient (001) and whether there is a need to alter the treatment. Tracking the patient's mood and symptoms can provide the opportunity to early detect a possible crisis and prevent the patient (001) from self-harm and suicidal acts.

### Digital Health Layer (1400)

The digital health layer (1400) comprises all the procedures required to track, analyze and treat the people who are seeking help to tackle their mental health issues. This layer comprises of all the application and devices developed to accomplish this aim, including several distinct processes:

1. Digital Intake management (102)

2. Digital symptom management (105)

3. Digital behavior management (108)

4. Diagnose management (103)

5. Digital screening management (104)

6. Report management (111)

7. Digital intervention/treatment management (107)

8. Digital crisis management (113)

9. Detecting inconsistency (109)

10. Alert management (110)

11. Clinical supervision (106)

The digital crisis management (113) is one of the main procedures considered in this layer to help patients (001) promote their well-being and refrain from committing suicide or any self-harm actions. This layer will provide adequate information about the symptoms that the patient (001) is indicating, the effects or potential side-effects of the treatment designed for them, changes in their behavioral patterns and also alerts the counselors (003) of the supporters (002) whenever the patient (001) is in an urgent need to undergo a digital crisis management (113) by tracking and screening their symptoms and mood. In addition, there is a built-in application in this layer to help all the aspects of treating patients (001) with mental health issues, namely counselors (003), supporters (002) and patients (001) themselves to be connected together so that they can support and help each other in various situations.

### People Interaction Layer

The last and the top layer in this model is formed by considering a network among various aspects of the problem of tackling a patient's mental health issue including: patients (001), supporters (002), counselors (003), and social community (004). These are the ones who provide information on the patient's situation and will track the patient's mood and symptom changes to identify the effectiveness of the treatment and whether the patient (001) needs to go through a digital crisis management (113). The data provided by these groups of people will be gathered and further analyzed in the digital health (1400) and data analysis (1300) layers to report them about changes in the patient's medical condition and alert them if the patient (001) requires an urgent crisis management (113).

### Use Case Diagram (101)

There are three agents considered in the proposed model who interact with the presented application and will deal with several sections of it, each designed and considered to do a specific work and accomplish a pre-defined purpose.
a) Patient (001): Patients (001) directly deal with almost all processes designed in the model, except for one or two procedures which will be done by counselors (003) and supporters (002). The procedures that patients (001) will deal with are as follows: Intake management (102), Diagnose management (103), Screening management (104), Symptom management (105), Intervention/treatment management (107), Digital behavior management (108), Detecting inconsistency (109) and Report management (111). Also, during the detecting inconsistency, provided that there is a disagreeing record found by the counselor (003), patients (001) will be notified about it. As a result, there is also an inclusion relationship between patients (001) and the alert management (110) procedure.
b) Supporter (002): Supporters (002) will be directly working with processes such as Intake management, Screening management (104), Symptom management (105) and Report management (111). In addition, due to the fact that supporters (002) will be affected by the alert management (110) procedure as they will receive an alarming notification about the seriousness of the situation that the patient (001) is dealing with and after Detecting inconsistency (109) in the patient's records, they are related to report management (111) by an inclusion relationship.
c) Counselor (003): The procedures of the presented model that counselors (003) will be dealing with are as follows: Intake management (102), Diagnose management (103), Screening management (104), Symptom management (105), Clinical supervision (106), Intervention/treatment management (107), Detecting inconsistency (109), Alert management (110) and Report management (111).

As it can be seen in the use case diagram (101) presented in this section, procedures might include other ones in their process, or be extended to the other processes. Such inclusions or extensions, comes from the fact that procedures might comprise of activities or actions that can be regarded as overlapped activities among different processes. For instance, to do a proper diagnosis, the process will need to consider the information gained from the intake management (102) as well as the screening management (104) procedures. As a result, the process of diagnosing (103) the patient's mental issue includes doing appropriate actions of intake (102) and screening management (104) procedures.

Similarly, the symptom management (105) may need to employ questionnaires filled by the patient (001) and the information gained from monitoring the patient (001) which is done by the counselor (003) and supporter (002). Thus, this procedure should include the intake management (102) and the screening management (104). The screening management (104) itself, will be done by tracking the patient's (001) symptoms, and consequently, includes the symptom management (105) procedure. In comparison, the clinical supervision (106) which is done by the counselor (003), will be required to track and monitor the patient's (001) mood changes, thus, only includes the screening management (104) as its co-operative procedure. During the clinical supervision (106), the counselor (003) will analyze the data relevant to the patient's (001) mental state such as any alteration in their mood and behavioral patterns, and will decide on a suitable and effective treatment plan to be developed in order to alleviate the patient's mental illness. As it was formerly described, treatment plans refer to the courses of actions which are suggested by the counselor (003) during the intervention/treatment management to help people with mental disorders and mental health issues deal with their problems.

Another procedure which needs to be done along with some other processes is called the intervention/treatment management (107). The mentioned procedure requires to be done in cooperation with the procedures such as symptom management (105) and diagnose management (103). As a result, when undergoing the intervention management (107), the procedures of symptom management (105) and diagnose management (103) should be taken into account as they are needed during the treatment management. Compared to the intervention management (107), while trying to detect possible inconsistencies among the patient's formerly recorded symptoms and the ones which are identified in the recent examinations, counselors (003) need to check and track the patient's behavioral patterns, and notify the supporters (002) about the inconsistency by an alarming notification. Hence, the process of detecting inconsistency (109) ought to include the digital behavior management (108), symptom management (105) and the alert management (110).

Finally, owing to the fact that preparing reports and recording analyzed data needs to be done by gathering information from several other procedures, the report management (111) should exclude some of the other procedures as its co-operative activities. In this regard, the report management (111) has an extended relationship with the digital behavior management (108), intervention/treatment management (107), the screening management (104), the symptom management (105) and the intake management (102) procedures, in addition to including the clinical supervision (106) that requires the documents gathered and formed by the report management (111) from various steps and procedures of the treatment.

### Comprehensive Flowchart (201):

The whole process of the proposed model begins with three main procedures that will be carried out in parallel. At the beginning of this flow when patients (001) want to start their treatments, a counselor (003) will be assigned to each patient (001) in order to help them fully perceive their condition and to develop a suited treatment plan in further processes. In the other steps, the patient (001) will undergo the digital behavior management (108) and the intake management (102) procedures. During the intake management (102), the patient (001) will have to complete some questionnaires relevant to his/her mental illness so that the counselors (003) will be able to better understand and diagnose the problems the patient (001) is struggling with. Digital behavior management (108) is a process by which the patient's behavioral patterns will be discovered using the data gathered by passive or active sensing approaches. These initial steps can be regarded as building blocks of the model, as the information obtained through them will be used for further analysis and preparing a well-developed treatment plan to enhance the patient's mental state and well-being.

Using the filled questionnaires by the patient (001) through the intake management (102) procedure, the person who is seeking help will go through several distinct processes such as “Report management” (111), “Symptom management” (105), “Diagnosis management” (103) and “Crisis Management” (113). First of all, the questionnaires asked from and completed by patients (001) will be formed as a reportal document for further analysis and recording the steps should be taken and their effects on patients.

In the symptom management (105) procedure, using the information gained in previous step, the indications and symptoms the patients (001) are showing due to the issues they are suffering from will be defined and then, the most prominent ones will be selected, tracked and monitored to identify the effects of the problem and the influence of the considered treatment on the patient's actions and performance. Then, any potential inconsistency in the symptoms recently reported by the patient (001) and the supporters (002) will be detected using the previously considered symptoms. If there are any incongruous records in the patient's symptoms, an alert will be reported to the counselors (003) to notify them about the disagreeing state of the reported symptoms. In addition to reporting the symptoms to the clinical supervisors and screening management (104) agents, the information obtained from the symptom management (105) chain of processes will also be employed to identify the possibility of a crisis in the near future. As a result, if the psychologists confirm any potential crisis occurring using their symptom management (105) information, the patient (001) should undergo the crisis management (113) procedure to preclude any possible disaster. Noteworthy, any action taken in this step, along with all the relevant information will be reported by the report management (111).

During the diagnosis management (103), the counselors (003) will diagnose the mental issues the patient (001) is suffering from based on the information provided by the patient (001) in the intake management (102). In this process, psychologists will make a decision about the patient's mental state and attempt to prepare a proper treatment plan, in later steps, to help them tackle their problems and promote their well-being and feelings. Then, the patient (001) will enter the screening management (104) and the clinical supervision (106) to be supervised by the clinicians and be monitored for any change in behavior and mood. Thus, the output of the screening management (104) will be used as an input for the symptom management (105) and vice versa, in addition to the clinical supervision (106). This will enable the psychologists to track the effects and side-effects of the diagnosis made in the former step. In addition, following the diagnosis management, counselors (003) will carry out an intervention management to develop a suitable treatment plan and track its influence on the patient's well-being using analyses of both the diagnose management (103) and the clinical supervision (106). Due to the fact that any change in the formerly developed treatment plan may cause effects on the behavior and mood of the patient, the outputs of this procedure will go towards the symptom management (105) and screening management (104). Also, resulting information and any step taken in this regard should be thoroughly recorded as the information is required for the future analysis. Consequently, there is also a link from the intervention/treatment management (107) and clinical supervision (106) towards the report management (111). Last but not least, the screened patient (001) might indicate a sign of likely crisis. As a result, the results of the screening management (104) will be used to detect any possible dangerous action such as self-harm or suicide for the crisis management (113).

Another output of the intake management (102) will go directly to the crisis management (113) procedure to identify whether the patient (001) requires any urgent need at the time to prevent any possible crisis from occurring. Provided that the patient (001) urgently needs help to be prevented from taking any self-harm action, the psychologists will help the patient (001) to go through a crisis management (113) procedure to enhance their well-being and mental state. Otherwise, if there is no immediate help needed, the symptom management (105) will be continuously carried on to detect any potential crisis in advance. On the other hand, when the patient (001) passes the crisis management (113) procedure and is claimed to be away from committing any dangerous actions, the process will continue from the screening and symptom management (105). Finally, the results gained through the crisis management (113) step will be recorded by taking the report management (111) step.

Digital behavior management (108) is a procedure to gather both passive data and the data from actively interacting with the patient (001) and supporters (002). The results of this process will not only be used to detect any disagreeing behavioral patterns in the patient's regular actions and to warn the professionals about the existing inconsistency and prepare appropriate reports, but also as an input to the intervention/treatment management (107) to identify any possible change in the treatment plan developed for the patient. This can help the professionals to adjust the treatment plan based on its influences on the patient's well-being. For instance, if an illness was misdiagnosed about a patient, the changes in symptoms severity can indicate that the associated psychologists should re-diagnose the mental illness. On the other hand, if the illness was correctly diagnosed but the treatment considered for the patient (001) has not been adequately successful, the professionals will be notified that the treatment plan needs to be adjusted.

### Digital Intake Management (102)

The digital intake management (102) procedure comprises two sub-processes designed for the patients (102-1) and their associated supporters (102-2). Many steps of this process are alike for both groups of patients (001) and supporters (002), except for the gained outcomes of this process.

Standard questionnaires have long been regarded to be one of the main tools in detecting illnesses, in particular issues relevant to mental health. In this regard, the digital intake management (102) procedure deals with questionnaires appropriate to the patient's mental state and begins with several parallel processes that should be done in the first step. To begin, suitable standard questionnaires are selected to be asked from and completed by the patient (001) (114). Regarding the supporters (002), appropriate standard question forms will also be selected for caregivers to examine their point of view of the mental issue the patient (001) is dealing with (115). This helps the counselors (003) trying to promote a sense of well-being in their patients, to consider both perspectives of the patients (001) and their supporters (002), leading to developing a more appropriate treatment plan. In addition, editing questionnaires will be designed and created by counselors (003) to adapt the former questionnaires according to the patient's mental condition to better suit them (116, 117).

Then, the results gained from the steps mentioned above will go through a process of designing and creating a proper flow of questionnaires to be done by patients (001) (118). This flow of question forms will assist the counselors (003) in perceiving the problems that patients (001) are suffering from and identifying the main causes of their mental illnesses in addition to understanding the best possible way to remedy them. Having this flow of questionnaires created and done by the patient, the results will be analyzed to understand the main aspects of, and potential solutions to the patient' mental illness (119). Noteworthy, all the analysis and question forms will provide an appropriate and thorough report in the report management (111) for further analysis in the treating process.

The analyzed data will be used to see if the patient (001) indicates any sign of necessity for undergoing follow-up questionnaires for the patient. In case the patient (001) needs any follow up question forms to complete, the procedure will be continued from the starting point by selecting standard questions and designing editing questionnaires for both patients (001) and their caregivers. On the other hand, when the patients (001) and their supporters (002) complete the flow of questionnaires relevant to their issue and there is no need for follow up questionnaires, the procedure will continue from the digital screening management (104), digital symptom management (105) and digital diagnose management (103). It means that after the mental issues were identified in this process, the patient (001) will go through a screening process and their symptoms will be monitored in addition to making a proper diagnosis based on the information gathered from these procedures. Such procedures help the counselor (003) identify and track any prominent symptoms as the treatment plan goes by as well as detecting any inconsistency in the patient's behavioral patterns in comparison to the former recorded symptoms. Any change in symptoms and behavior will lead to alterations in former diagnosis and the developed treatment.

Regarding the supporters (102-2), the results from the data analysis process (119) will be used to see whether there is a need for follow up questions or not. Similar to the flow design for the patients (102-1), when supporters (002) need to undergo a follow up examination, the procedure will continue from the starting point, meaning selecting the suitable questionnaires (114-117). On the other hand, if the supporters (002) do not indicate any need for further questionnaires, their procedure will continue with beginning from the screening management (104) and the diagnosis management procedures (103).

### Digital Symptom Management (105)

The digital symptom management (105) procedure comprises of two distinct chains of steps, each associated with either patient (105-1) or their supporters (105-2) which are explained respectively in the following.

### Digital Symptom Management—Patient (105-1):

The symptom management considered for patients (001) (105-1) begins with detecting what symptoms the patient (001) is indicating at the moment (120). Any change in symptoms relating to mental health can be detected and identified by analyzing certain patterns of smartphone use. For instance, the sleep disturbance might indicate serious depression and suicidality among patients. Moreover, choice of music and communicative language, in addition to facial expressions received from camera sensors can help counselors (003) in understanding the mental state of the person under their treatments and become aware of possible suicidal thoughts. Therefore, various smartphone sensors and wearables—like mobile phone's camera, microphone, application use, light sensors as well as smart wearable gadgets which detect heart rate, body motion, movement, changes in location and alterations in blood pressure, sleep and things as such—might be helpful in this regard, as a result of gathering real-time information about the patient's mood change, depressive thoughts and context aware monitoring of improvement made in their treatments.

Then, from all the indications obtained by the previous step, the prominent ones will be selected for further analysis (121). This means that by focusing on the most crucial symptoms, counselors (003) might be able to develop a more suited and efficient treatment plan for the person who is seeking help. In addition, not all behavioral patterns and symptoms are related to the serious illness the patient (001) is suffering from. As a result, such irrelevant symptoms might raise the risk of misdiagnosis or over-diagnosis. In the next step, the symptoms will be tracked (122) and monitored (123) to distinguish any change in them, whether being downscaled or amplified. This can help to adjust the treatment plan to better cover the patient's conditions and medicine's possible side effects in addition to identifying any potential misdiagnosis of the patient's illness.

The result of this process will be going through several procedures to further assess the patient's treatment efficacy and to notify the professionals and supporters (002) in times of high risk for a crisis. In this regard, the symptoms will be monitored by a counselor (106) along with the screening management (104) procedure. Following this, recent symptoms will be checked according to the former indications and information received from the symptom management procedure which is associated with supporters (105-2), to detect any incongruous between them (109). Provided that there is any inconsistency between the previous and later symptoms, counselors (003) should be notified by an alert through the alert management (110) module.

Also, the result from monitoring the patient's symptoms will be used to prepare an appropriate report (111) as well as to manage the treatment and required interventions to promote the patient's well-being (107). The mentioned procedures are important since any change in symptoms or showing any potential side effects will influence, whether significantly or marginally, the treatment plan developed for the patient. Finally, if the patient (001) is suffering from a serious condition by which they might face any possible crisis, the counselors (003) will be notified by tracking and monitoring (122-123) the symptoms the patient (001) is indicating, thus, they will check if there is any need for crisis management (113). In case there is such a requirement for developing a crisis management plan, the patient (001) will undergo the appropriate procedure (113). Otherwise, whether there is no need for such a procedure or the patient (001) has passed the crisis management (113), the steps of symptom tracking (122) and symptoms detection (120) will continue respectively until they indicate that the patient (001) might encounter a crisis soon.

### Digital Symptom Management—Support (105-2):

The symptom management procedure associated with supporters (105-2) is generally akin to the one for the patients (001) (105-1) except for a marginal difference in detecting a need for a crisis management (113). In the first stage, the symptoms the patient (001) indicates are detected (120), and then among all the detected symptoms, the ones that are considered to have a higher priority in comparison to the others are selected to be tracked and monitored in further steps of the process (121).

Similar to the patient's flowchart, the information formed by monitoring symptoms (123) will go to several distinct procedures to be further analyzed. In this regard, the results gained from monitoring the symptoms (106) along with the information received from the screening management (104) associated with supporters (002) will be monitored by a counselor (003) to identify any possible change in symptoms (106). Furthermore, using obtained information from both patients' (001) and supporters' (002) perspectives, any incongruous symptoms will be detected (109) to notify the psychologists about them by an alert to adapt the treatment plan more effectively (110).

Finally, the monitored symptoms will be used in writing and preparing reports (111) as well as in identifying any requirements for crisis management (113). In this case, if the supporter (002) notifies that the patient (001) needs to be undergone a crisis management, such request is also checked according to the patient's crisis management procedure to ensure that the patient (001) is certainly potential to face a crisis rather than a false alarm. In the latter case when such a request has just been received from supporters (002) rather than both parties, an alert will be done by the alert management module (110) to notify the professionals of the possible false warning. Such caution is important because any incorrect intervention in the patient's treatment might interfere with the whole treatment with deleterious effects on the patient's improvement. If the patient (001) is detected to be facing a crisis, the crisis management procedure (113) will be carried out. Otherwise, if supporters (002) do not indicate any need for crisis management in the first place, the symptoms will be continuously tracked (122). In addition, when a patient (001) passes the crisis management procedure, the procedure will begin from the starting step, in other words, the symptoms detection (120).

### Digital Crisis Management (113):

Employing digital tools is expected to ease the procedure of crisis management (113), helping counselors (003) as health professionals to monitor the patients' medical states more often than face-to-face interviews and interactions. Having provided many opportunities in the medical world, this promising approach may assist psychologists in developing more appropriate treatment plans. In this regard, making use of digital instruments in crisis management (113) can help professionals preclude a threat occurring promptly by tracking the patient's mental health and using gathered and integrated information from the patient, supporter (002) and the assigned counselor (003).

The digital crisis management (113) procedure proposed here generally comprises of two main parts: the flow of the process arranged for patients (113-1) and the flow arranged for their supporters (113-2).

### Crisis Management-Patient (113-1):

When a patient's mental condition is endangered and the patient (001) is recognized as needed for crisis management, several steps must be taken. In this regard, the patient's location will be continuously tracked (130) and identified using an in-built module in the crisis management procedure. This tracking followed by checking whether the patient's location is safe or unsafe (133), using the information provided in both patient (001) and support (002) application, in addition to the information received from the digital behavior management (108) as inputs to the crisis management procedure (113-1). As a result, the patient (001) being present at a place which has been considered unsafe according to the gathered information, is regarded as an initial sign of potential crisis in a short time. The need for undergoing the crisis management is also indicated by three other sources: symptom management (105) and screening management (104) procedures along with the counselor (003) assigned to the patient (001) at the beginning of the treatment.

The symptom management (105) is a process by which the patient's symptoms for mental health problems are detected, tracked (122) and monitored (123) to investigate the effects of the treatment developed for the patient, identifying any potential inconsistency in the patient's symptoms (109) and to notify the counselors (003) whenever there is a potential risk of crisis. Both patients (001) and supporter's (002) applications are considered sources of information in this regard, and the integrated information gained from them help the professionals decide whether the patient (001) is definitely required to undergo the crisis management (113). The screening management (104) procedure may provide another early sign of need for running a crisis management, especially when issues are not clearly identified in early interviews with the patient (001). Through the screening management (104), the patient's mental state is examined using an appropriate flow of either paper-based or digital questionnaires. Analyzing the results obtained from these questionnaires helps the professionals to identify whether the patient (001) requires an urgent crisis management or not. All these sources can help counselors (003) to perceive whether the treatment plan developed for the patient (001) has been successful in treating and moderating the illness the patient is suffering from or the patient's condition has been exacerbated.

Consequently, the information received from these sources goes through several distinct steps ranging from identifying the helpful tools (124) for feeling better to considering supportive people (128) and being linked to them in order to develop a so-called “safety plan” for the patient. Thus, in hours of experiencing severe mental issues, patients (001) can employ these tools to promote their feelings and well-being. As a result, it can be claimed that using the identified tools can preclude the patient (001) from suffering a severe mental illness that could be considered a crisis.

In the first stage of this flow, initial steps for developing a mental crisis management plan are taken, like identifying instruments that might help the patient feel better (124), early warning signs according to the patient's behavior patterns and symptoms (125), and the stressors (126) that typically pressure the patient. In addition to identifying essential information to be used in the further process, the patient's application is also linked to the application associated with the supportive person (supporter (002)) (127). In addition, some people whom the patient (001) trusts are added to the list of calls to be contacted when necessary by the patient (128). As well as considering the places that are regarded as the patient's safe locations (129).

After considering all the potential safe locations and tracking places where the patient (001) is at the moment (130), the patient's current location is tracked according to whether the location is safe or unsafe (133). This decision is made based on the list of possible safe locations obtained from the patient's application and the list of unsafe locations received from the support safety application (137). The process of monitoring where the patient (001) is will continue until it is confirmed that the patient's location is regarded as an unsafe place. In this case, an urgent alert needs to be done by the module provided for the alert management (110) in this process, in order to notify the professionals and supporters (002) to prevent any potential crisis from occurring. As long as the process of monitoring is going on, any necessary reports should be done according to regulations established in the report management (111).

Parallel to the location tracking process, the information gained from the steps of “identifying feel better tools” (124), “early warning signs” (125), and primary “stressors” (126) are checked in (131) and then analyzed in the “check-in analysis” (132). The latter process has two outputs, and one of them goes through the “Report management (111)” to prepare the appropriate reports for further analyzing and tracking the treatment. The other output is regarded as a factor indicating whether urgent help is needed for the patient's health and well-being. If any crucial need is necessary, the patient (001) requests an urgent call (134). In this case, the alert management (110) module will be activated to notify the professionals and the supportive people and also the system proposes to patients (001) to call 911, chat line or hotline. On the other hand, if the patient (001) does not need any urgent help at the moment, they are suggested to call their trustful people (135) or use so-called “feel better tools” (136) to promote their feelings and well-being based on the results of check-in.

### Crisis Management-Support:

When a patient (001) has to go through a crisis management (113), their supporters (002) are also involved. In this case, like what is done for patients (001) in the flow associated with them (113-1), useful tools for feeling better (138), early warning signs of crisis (139), and essential stressors (140) are identified for patients (001) by the supporter (002). Next, employing this information and using the patient's risk profile (152), the information is checked about the patient by the check in process in support application (141). As a result of this process, the suitable rooms in the internal social network (142) and the proper guidelines for the patient's well-being are proposed (143). Then, in parallel to the two steps mentioned above, the output of the check-in process is also analyzed (132) based on the patient check-in results (131) and support check-in results (141) and a suitable report is done about its analysis in the report management (111).

The other part of the flow associated with supporters (002) has something to do with identifying the safety of the patient's location. In this section, safe and unsafe locations for the patient (001) are added to the list of potential places where the patient might be (144, 145). Then, the safe locations are compared (146), and the required report is prepared for them (111). Secondly, the patient's location is monitored (147) based on the information associated with safe and unsafe locations along with the interpreted results obtained from the patient's behavior pattern tracking (108) through passive mobile/digital sensor data gathering and actively interacting with the patient (001) and their supporters (002). Consequently, if the patient (001) is in an unsafe location or when the patient (001) requests an urgent call (134), an alert need to be done by the alert management (110) module, and then the supporters (002) should receive the appropriate notifications of this (149). Otherwise, monitoring the patient's location (147) will continue until the patient (001) turns up in a place that is regarded as unsafe.

Finally, the supporter (002) can request the status to be updated (150), and this regular updating of the patient status can help the support be up to date with the patient's mood. Thus, they might be assured that they can promptly prevent any potential crisis so that the patient's well-being and the prevention of any possible self-harm behavior and suicide attempt might be guaranteed. Again, the result of this status being updated goes through the report management (111) to prepare any required report for further analysis and tracking of the patient's treatment.

### Digital Diagnose Management (103):

Having the patient (001) gone through the intake management (102), it will provide required information for the counselors (003) to use in the diagnose management procedure (103). In the first stage, this process will begin with two types of clinical and automatic diagnoses, with the codes of (153) and (154), respectively.

The clinical diagnoses (153) will be done by going through the several distinct procedures such as the screening management (104) procedure designed in the proposed model, the clinical supervision (106) done by counselors (003). Noteworthy, in the case of clinical diagnoses (153), the data gathered in the clinical supervision (106) will be used in the intervention/treatment management (107) for further analysis.

Another class of diagnoses is named the automatic diagnosis (154) which will be followed by the patient (001) undergoing the screening management (104), clinical supervision (106) by the doctors in the clinical environment and finally, going through the intervention/treatment management procedure (107) directly.

### Digital Screening Management (104):

The digital screening management (104) begins when there are indications received from the digital diagnosis management (103) and the digital symptom management (105) procedures, signifying that the patient (001) needs further examination which mainly is done by asking them to complete more questionnaires. As a result, the digital screening management (104) procedure begins with selecting standard questionnaires for patients (001) and supporters (002) associated with them, (114) and (115), in turn.

Then, the selected questions will be employed to design and create a suitable flow of questionnaires (118) to be used to examine the patient's mental health state. Next, the counselors (003) will analyze (119) the results from the above step along with the received information from the intervention/treatment management (107) and the ultimate results of this data analysis will be used to prepare a report (111) together with information about the designed and asked questionnaires, to record everything during the treatment in case they are needed in the future.

The analyzed data will also be employed for two other purposes to detect any possibility for experiencing a near crisis and consequently any need for crisis management (113), in addition to identifying if the patient (001) needs further treatment monitoring. In the first case, either there are no such requirements for the crisis management (113) or after the patient (001) has passed that procedure and there is no further need for this process to be done, the patient (001) will go back to identifying if further treatment monitoring is needed. Provided that more treatment monitoring is needed for the patient, the procedure will continue from the starting point of the digital screening management (104). On the other hand, if there is not such monitoring required to be done, the patient's treatment will go on by beginning the clinical supervision (106), the digital symptom management (105) and the treatment management procedures (107).

### Digital Intervention and Treatment Management (107):

The need for the patient (001) to undergo a digital intervention/treatment management (107) procedure is indicated by several steps in the treatment, namely the digital diagnose management (103), the digital behavior management (108) and the clinical supervision (106), and by interpreting the information gained in these procedures which are regarded as inputs to this process. After being noticed in these processes that the patient (001) needs to go through the treatment management (107), the process associated with the treatment of the patient (001) will redirect to this procedure so that counselors (003) must take several specific actions.

On the whole, there are two distinct classes of steps in the flow of the intervention/treatment procedure (10), regarding the two types of treatments used to help those who are dealing with mental issues:

### Clinical-Based Intervention/Treatment (155):

Clinical-based treatments are the first class of treatments, considered in this flow of intervention management (107) in the proposed model, which will be explained here. In the first stage of this chain of steps, the appropriate reports will be generated based on the information gained as the inputs of this procedure from the diagnosis (103) and behavior management (108) in addition to the clinical supervision (106) done by counselors (003). Then, these gathered data and prepared reports (157) will be analyzed (119) to see if the patient (001) might experience a crisis soon, and consequently needs to undergo a crisis management. If the counselor (003) decides that the patient (001) requires such a process, the treatment will continue by the crisis management procedure. On the other hand, either the patient (001) does not seem to be about to face a crisis or they have passed the crisis management procedure (113) and there is no need for that, treatment will continue from the starting point of the intervention/treatment procedure (107).

### Technological-Based Intervention/Treatment (156):

The technological-based treatments will begin with several steps that ought to be taken. These processes might all be needed to be applied to the patient. Although, in some cases, some of them can be discriminately selected according to the patient's medical condition.

In this regard, the patient (001) might need to undergo processes such as the ones listed in the following to gather vital information about the efficacy of the treatment formerly developed for the patient (001) by counselors (003):
1) Psychotherapeutic mindfulness and ACT (158): this process will be followed by the applications (169) being checked by the counselor (003).
2) Psychotherapeutic interventions (159) that are followed by the doctors inspecting the patient's chatbot (167), applications (169) and smart clothes (170).
3) Gamified interventions (160) that will continue with checking the patient's applications (169), smart clothes (170) and extended reality (171).
4) Preventive interventions (161). This process will be done by investigating the patient's applications (169), smart clothes (170) and extended reality (171).
5) Psychotherapeutic CBT (162): in this step, the counselor (003) will check the applications (169), chatbot (167) used by the patient (001) and their extended reality (171) to gather all the related information.
6) Measurement and follow-up (163) that will be done by examining all of the patient's chatbot (167), applications (169), smart clothes (170) and extended reality (171) related information.
7) Supportive interventions (164) that are followed by checking the patient's chatbot (167), the built-in social media (168) in the application, smartphone applications (169), smart clothes (170) and finally the patient's extended reality (171) to gather all the relevant data for future analysis (119).
8) Skills training (165): This process will go on by examining the patient's applications (169) and extended reality (171) to analyze the data gathered from them (119).
9) Behavioral intervention (166) that will be done by the investigation of the patient's used chatbot (167) and applications (169) to track the patient's behavioral patterns and possible changes in mood and behavior.

Then, the information gained by examining various types of the provided facilities used by the patients (001) to track their moods, symptoms and behavior, will be analyzed (119) in the next step to see whether the patient (001) indicates any need for a crisis management procedure. In case the patient (001) is supposed to face a crisis in a short time, the clinician (003) ought to assist the patient (001) to pass the potential crisis by considering a crisis management plan, or as it is usually called, a safety plan. Whether the patient (001) needs to undergo any crisis management (113) or not, the intervention/treatment management procedure (107) will repeat for them, maybe with different prescriptions in each period. Noteworthy, the data analyzed in this flow will also be used for further monitoring and analysis in the symptom management (105) procedure in addition to the report management (111) to prepare a comprehensive report of the patient's improvement and reaction to the treatment developed for them.

### Assignation a Counselor (112):

To help the patient (001) promote their feelings and support them in every step of their treatment, a counselor (003) should be assigned to the patient (001) in the first place. In this regard, there is a procedure designed for assigning a counselor (003) to the patient (001) in the proposed model.

The assigning counselor (112) begins with checking if the patient (001) is less than 18 years old or not. If the patient (001) is older than the age limit considered in the model, they are allowed to select a counselor (003) for themselves who helps them promote a sense of well-being (172). Otherwise, if the patient's age is lower than 18, their associated supporter (002) should choose a clinician for them (173). Then, there are several options provided for both patients (001) and supporters (002) which are identical and can be used by either of them. The only exception here has something to do with the payment management (175), which is specialized to be used by patients (001) who are above 18 years old, and have chosen their doctors themselves. That is to say, when a patient has the requirement of the age, which is the condition when the patient is aged above 18, they are in charge of the financial consideration of their treatment and assigning counselor (003). Otherwise, their supporter (002) has to deal with these considerations.

The following actions which are allowed to be taken by both patients (001) and supporters (002) after selecting their counselor (003) (172, 173, respectively), are as follows. First, they can view other people's comments and opinions (174) about the counselor (003) to become familiar with their way of behaving with patients (001) or what approach they usually tend to choose to treat patients (001) with similar conditions. Then, their selected counselor (003) will be linked to the application developed for the patients (001) suffering from mental illnesses (176). After being connected to each other, the patient (001) is able to make an appointment (177) to meet the doctor face-to-face and have an interview or be virtually interviewed by the chosen counselor (003) through online meetings facilities (178).

There is also a built-in messenger (179) provided for the patients (001) and their supporters (002) to chat with their chosen clinician and call them to notify them about changes in symptoms or mood, in addition to asking questions whenever they feel an urgent need to do so. Furthermore, they are allowed to leave comments (181) and score their own counselors (180) to help other patients (001) and supporters (002) in their selecting counselors (003). Finally, provided that the patient (001) and their supporter (002) are not satisfied with the treatment developed by their selected doctor or if they have had to tolerate any inconvenience in the process of being treated, they are allowed to change their counselor (003) and choose another one who might help them better and more effectively (182).

### Digital Behavior Management (108):

Digital behavior management (108) begins with gathering relevant data by employing the approaches of passive data collecting (183) (using patient's mobile/digital sensor and tracking their behavior pattern), and active sensing (184) (by considering interactions with patients (001) and their supporters (002)) in parallel. In the next stage, the gathered data obtained from the past two steps will be checked and put together (185), followed by data cleaning (186) and data analysis (119) steps. Then, the results gained from the acquired information will be interpreted (187) to identify and track the behavioral patterns associated with the patient (001) and their supporters (002).

Next, the results and their analytic interpretation (187) will go through several appropriate procedures such as detecting inconsistency (109), report management (111) and crisis management (113), in order to take the proper action based on the patient's indications in behavioral patterns. For instance, there should be an analysis to identify whether the patient (001) is at the high risk of facing a crisis. In this case, the analytic results should go to the crisis management (113) module to examine the severity of the situation and to develop a crisis management plan to promote the patient's well-being with adequate efficacy. Furthermore, there should be a report of any symptom and behavioral pattern associated with the patient (001) in addition to the relevant information about the treatment's effects on the patient's symptoms, that is carried out by the report management (111) module.

### Report Management (111):

As a matter of fact, preparing comprehensive reports by recording analyzed data, any slight changes in the patient's mood and symptoms can play a significant role in assisting the counselors (003) to track the effectiveness of their patients' treatment, and their improvement and possible side-effects. As a result, there should be a section devoted to preparing and managing reports in any model that aims to help patients (001) dealing with mental health illnesses, promote a sense of well-being. Consequently, the report management procedure (111) designed in the proposed model, gathers information from all the procedures and makes complete reports by considering all the prominent data gained that will be useful in future steps of the patients' treatment.

To begin with, there are two approaches considered in the presented report management (111) procedure, regarding preparing reports by counselors (188) or system engines (190). Firstly, the information gathered in the digital diagnosis (103) and intake management (102) procedures will be used to scribe and draft the reports (188). In addition, the videos and audio content received from the patient (001) during the process of treating them will be tagged with suitable labels. Next, the information from the previous step joined with the data gathered from monitoring the patient, tracking their behavioral patterns and changes in mood and symptoms in the screening (104) and symptom management (105) procedures, are employed to prepare thorough clinical reports by counselors (189).

On the other hand, there is a systematic report engine (190) provided in the presented model to prepare spontaneous reports for the patients (001) under treatment, their supporters (002) in addition to their assigned counselors (003). In this regard, relevant and useful information will be collected from all the distinct procedures, namely intake management (102), digital behavior management (108), screening management (104), intervention/treatment management (107), clinical supervision (106), symptom management (105), social network management (191) and crisis management (113), and then will be given to the system report engine to prepare appropriate reports such as intake report (192), digital behavior report (193), screening report (194), intervention/treatment report (195), counselor (003) prepared report (196), symptom management report (197) and social network report (198), that will be used to examine the effectiveness of the developed treatment plan and track the patient's mental state improvement. Ultimately, all the prepared reports will be used by counselors (003) in the clinical supervision (106) to ensure the suitability of the treatment plan developed for the patient.

### Social Media (168):

To help different aspects of dealing with a mental illness—namely the patients (001), supporters (002) and counselors (003)—to be more connected to each other, there is a built-in social network named “Socialin” (168) provided for them in their applications, that creates the opportunity of having more frequent and direct communications. This not only will assist counselors (003) in tracking the patient's improvements by interviewing patients (001) more regularly, but also helps patients (001) and supporters (002) themselves to know what should be done according to their current condition. As a result, the treatment's efficacy may not be inflicted by taking incorrect actions in serious circumstances.

Due to the fact that there are three distinguishable yet entirely related aspects, regarding the treating of a patient dealing with a mental illness, three distinct flow are considered and designed in the proposed model to illustrate different options and processes provided for them:

### Social Media-Patient (168-1):

When patients (001) create their profiles in the social network (199), many options are provided for them to use in order to promote their feelings in addition to sharing their thoughts and ideas with other ones who might be experiencing similar situations. First, patients (001) will be provided with the list of club houses which they have already joined (204), and they are allowed to send requests to join the other club houses (205) that have been created by counselor (003). In this case, provided that their supporter (002) have permitted them to do so, patients (001) are able to join the counselor's club house (207) and then they can insert various contents like text or voice messages, and edit or delete their previously sent textual contents (209), until they are still present in that club house and has not left it. On the other hand, if they are prevented by their supporter (002) from joining the club house, the patient (001) will receive a “not permission” message to be informed of not being allowed (208). Both this message and the content sent by the patient (001) in the other case, will be checked and analyzed by the content management analysis (217) and an appropriate report will be prepared for it in the report management procedure (111).

Moreover, patients (001) can create, edit, delete or post the therapeutic content to publish in the explore designed in the application (206), in addition to edit or delete their public profile (211), sharing/unsharring their favorite music, photos and videos as well as their daily mood-change records and check-ins in their public profiles (212) so that other users are able to see them. Provided that the patient (001) is not willing to share their favorite contents and check-in results with other ones publically, they can choose to either share/unshare their contents and daily check-in records just with their own followers (213), so no other user can view their content. On the other hand, if they have decided to post them to the explore section of the application, they are provided with the option to send their music, video and photos to the explorer, and edit, delete their formerly posted contents (214). Otherwise, they are also provided with the option to send their contents, including music, video, their daily check-in and photos, to their followers, in addition to being able to edit or delete them as they wish (215).

Patients (001) are allowed to either follow or unfollow other users who are present in the built-in social network of the application (216). Such activities will be done anonymously so that no one will be emotionally influenced in case they have been unfollowed or followed by others. Finally, to give the users similar feelings of using typical social networks, patients (001) can like/dislike comments (200) or leave comments about the posts created by others (201). They are also provided with recommendations about the posts they might be likely to be willing to see, based on their previous activity, favorite and similar contents (202) in addition to viewing the recommended users identified by their common favorites (203). It should be mentioned that any posted content on the social network will be thoroughly checked by a content management (217) to ensure that no harmful content would be passed to other users employing the application.

### Social Media-Supporter (168-2):

After creating a profile (218), supporters (002) have access to a variety of options. First of all, they can view the list of club houses they have joined earlier (219), and are allowed to join those club houses that have been created by the counselor (220). Having joined a club house, supporters (002) are able to insert new text or voice messages as well as editing or even deleting their previous text messages, during the time that they are still a user of that club house and have not left it (209). They can also edit or delete their profiles (223) and are provided with content or user recommendations based on their favorites and the similarity found between posts and common favorites among users (202 and 203, respectively).

Another option considered for the supporters (002) is the ability to create subject oriented online discussion rooms (221), in addition to being able to join other subject oriented rooms (222) created by other supporters (002). Then, supporters (002) who are present in an online room, can send various types of media such as text, voice and photos, and edit or delete their previously sent messages and files (224). They are also able to like or dislike comments posted on social media (200) and see other comments and users like/dislike scores (225). These scores can help them decide on choosing their counselor (003) or provide them with the opportunity of creating connections with helpful supporters (002).

Finally, like the process which controls contents before distributing them in the social networks designed for patients (001) and counselors (003), here, there is also a content management analysis (217) which examines any content prior to their insertion to the social network. In addition, to gather supporters' (002) activities and information in the social network and form them into useful to be analyzed in further process, complete and appropriate reports will be produced using report management procedure (111).

### Social Media-Counselor (168-3):

The social media flow designed for counselors (003) begins with creating a profile (218) using required documents and information that the application asks them to prepare for registration. Then, they are provided with several options to use, such as viewing the list of club houses and creating, editing or deleting club houses (226), being able to chat and send messages (228) with patients (001) and their supporters (002) privately, and edit or delete their own profiles (223) whenever they are willing to. Furthermore, counselors (003) can join other councilors' clubhouses to participate in their debates (227). In this regard, they are allowed to insert different contents such as text or voice messages and are able to edit or delete the textual content they have sent before (209). This option will be available for them until they are present in that club house and have not decided to leave it (210).

Another feature provided in the built-in social network in the proposed application for counselors (168-3), is to create social rooms and forums to gather people around a specific subject to discuss about (221), or to join the rooms already created (222) by other supporters (002). Creating the subject oriented rooms will help the counselors (003) and others who are present in that room to give their opinions and share their experiences and thoughts around a chosen subject, that consequently might help them to see the problem from a different perspective or become aware of a potential solution. In these online rooms, a counselor (003) can send, edit or delete different contents such as text, voice or photos (224), leave comments or like/dislike others' comments (200), and view the score given by others to comments and users (225).

It is noteworthy to mention that all the provided contents in these online rooms and club houses will be analyzed by the content management analysis (217) to prevent any harmful content that might have deleterious effects on the patient's treating process or on other ones, to be distributed among the social network users. Then, appropriate reports related to the activities done in these forums and all the useful information about them will be prepared in the report management (111) procedure for future analysis.

While preferred embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments of the invention described herein, may be employed in practicing the invention. It is intended that the following claims define the scope of the invention and that methods and structures within the scope of these claims and their equivalents be covered thereby.

1- A digital mental health process and structure thereof, comprising distinct architecture of a fog layer, a cloud layer, a data analysis layer, a digital mental health layer, and a people interaction layer; wherein each one of said layers interact with one another, and wherein said Fog layer comprises computational and handling aspect of said digital mental health process; wherein said Cloud layer comprises storage and real-time services for said structure; wherein said Data analysis layer comprises data acquisition, data cleaning and data analysis of information received from said people interactive layer; wherein said Digital health layer comprises a group of interactive connected applications, where each one of said applications comprises steps helping patients dealing with mental issues, tracking their mood and behavior; and wherein said People interactive layer comprises steps connecting said patients to their supporters, counselors and social community.

2- The process of claim 1, wherein said fog layer is located behind said cloud layer and comprises of several fog nodes providing computational power, device controlled capacity, storage and connectivity services, and further comprise I/O devices, switches and gateway computers; and wherein said structure and process thereof having the following steps of:
a) receiving extensive patient's medical and non-medical related information from different sources;
b) analyzing said information; and
c) providing real-time reactions and answers; wherein steps (a) through (c) are performed in anyone and all of said fog layer, cloud layer and digital mental health layer.

3- The process of claim 2, wherein said cloud layer comprises several cloud servers performing the steps of:
d) monitoring and controlling security of fog data received from said fog layer;
e) processing and storing said monitored fog data;
f) processing and storing analyzed data received from said analyzing layer; and
g) providing real-time service and answers for said patients.

4- The process of claim 3, wherein said data analysis layer comprises three main sections of data acquisition, data cleaning and data analysis of mental data and information of mental health condition of said patients, received from said digital mental health layer; comprising the steps of:
h) cleaning and removing any noise from said mental data; wherein said cleaning comprises fixing, eliminating incorrect, corrupted, incomplete, duplicate and incorrectly formatted data;
i) analyzing said cleaned mental data;
j) indicating any changes in the mood and behavioral patterns, frequency of suicidal thoughts and ideation, risk of suicide in said patient;
k) forming and sending a comprehensive reports to said patient, supporters, and counselors; and
l) tracking improvements made in treating said patient by said counselors; and
m) altering said treatment based the results in step (l).

5- The process of claim 4, wherein said digital mental health layer comprises digital Intake management, digital symptom management, digital behavior management, diagnose management, digital screening management, report management, digital intervention/treatment management, digital crisis management, detecting inconsistency, alert management and clinical supervision sections; and wherein said digital crisis management comprises the step of:
n) gathering symptoms and any changes in behavior of said patients, indicating effects or potential side-effects of said treatment for said patients;
o) alerting said counselors and said supporters;
p) performing an urgent digital crisis management; and
q) connecting said counselor, said supporter and said patients with each other via a built-in application; and
r) helping and supporting said patients promote their well-being and refrain from committing suicide or any self-harm actions via said digital crisis management and said built-in application.

6- The process of claim 5, wherein said people interactive layer, comprises the step of:
s) creating a network between said patients, said counselor, said supporters and social community;
t) tracking said patients' mood and symptom change and said gathered data from said digital mental health and said data analysis layers;
u) identify effectiveness of said treatment and the need to start said digital crisis management.

7- A digital management mental health plan, comprising the steps of:
a) assigning a counselor to each patient;
b) helping them fully perceive their condition and developing a suited treatment plan;
c) performing a digital behavior management and intake management procedures on each of said patient;
d) diagnosing and understanding the state of mental illness of said patient in said intake management procedure, based on data from filled questionnaires gathered by said counselors;
e) discovering behavioral patterns of said patient using said gathered data by passive or active sensing approaches;
f) analyzing and preparing a well-developed treatment plan to enhance said patient's mental state and well-being;
g) performing additional analysis of report management, symptom management, diagnosis management and crisis management on said gathered data;
h) providing a report document of said gathered data; and
i) defining indications and symptoms of mental and physical issues of said patient;
j) selecting the most prominent ones of said indications and symptoms;
k) tracking and monitoring the effects and influence of a considered treatment for said selected symptoms;
l) detecting any inconsistency in said selected symptoms reported by said patient and said supporters;
m) alerting and reporting any incongruous records in said patient's symptoms to said counselors;
n) employing information obtained said symptom management procedure and identifying occurrence of a crisis for said patient and therefore performing said crisis management procedure to preclude any possible disaster; and
o) reporting all the actions in steps (a) to (n) and all of said gathered data to management.

8- The digital management plan of claim 7, wherein further comprises the steps:
p) diagnosing said patient's mental issues in said diagnosis management procedure, based on said gathered data from said intake management;
q) preparing necessary treatment plan for said patient;
r) entering a screening management procedure and clinically supervising and monitoring any changes in the mood and behavior of said patients; wherein an output of said screening management procedure will be used as an input for said symptom management procedure and vice versa;
s) tracking effects and side-effects of a diagnosis made based on steps (a) through (r);
t) carrying out an intervention management procedure and developing and tracking the influence of a treatment plan procedure by using analyses of both the diagnose management procedure and said clinical supervision procedure; wherein a treatment output of said treatment plan procedure is considered an input to said symptom management and said screening management procedures; and
u) recording and reporting information from said intervention/treatment management procedure, said clinical supervision procedure in report management.

9- The digital management plan of claim 8, wherein another output of said intake management procedure directly feeds into said crisis management procedure, identifying an onset of occurrence of a crisis; and prompting said patient to participate in said crisis management procedure and therefore preventing any harm to said patient.

10- The digital management plan of claim 9, wherein continuously running said symptom management procedure and therefore detecting occurrence of any crisis in advance; the results gained through said crisis management procedure is recorded in said report management procedure.

11- The digital management plan of claim 10, wherein in said digital behavior management procedure passive data and the interacting data between said patient and said supporters is gathered; wherein a result of said digital behavior management procedure is inputted to said intervention/treatment management procedure; wherein said result detects disagreeing behavioral patterns in said patient's regular actions and reports a warning of a change in treatment plan to said supporter.

12- The digital management plan of claim 11, wherein said digital intake management procedure comprises two sub-processes designed for said patient and their associated supporters;
comprising the steps of selecting standard questionnaires to be asked from and completed by said patient and selecting appropriate standard question forms for supporters; gathering both perspective of said patient's mental health and their said supporters'; wherein said counselors edit and design questionnaires according to said patient's mental condition.

13- The digital management plan of claim 12, wherein answers to said questionnaires provides a flow of new and customized questionnaires designed for said patient specifically and therefore provides better understanding main aspect and solution for said mental health issue/illness; wherein a report of all the aforementioned steps is provided via said report management procedure, for further analysis and treating process.

14- The digital management plan of claim 13, wherein said digital symptom management procedure comprises of two distinct chains of steps, each associated with either said patient or their said supporters; wherein said digital symptom management procedure comprises the step of detecting what symptoms said patient is indicating, selecting the prominent indicators for further analysis; wherein further comprising the step of tracking and monitoring said symptoms change; and therefore adjusting said patient's treatment plan; wherein recent symptoms is checked according to said indications and information received from said symptom management procedure associated with said supporters; detecting any inconsistency and therefore notifying said counselor via an alert through an alert management module.

15- The digital management plan of claim 14, wherein said digital crisis management procedure comprises of two main parts: a process flow arranged for said patients and an arranged flow for their said respective supporters; wherein said digital crisis management procedure further comprises an in-built tracking and identification module, receiving information from digital behavior management procedure as input.

16- The digital management plan of claim 15, wherein said symptom management procedure comprises the steps of detecting, tracking and monitoring said treatment developed for said patient, identifying any potential inconsistency in said patient's symptoms and notifying said counselors; wherein said screening management procedure provides another early sign of need for running said crisis management.

17- The digital management plan of claim 16, wherein a digital diagnosis procedure comprises the steps of performing screening management procedure; gathering data from said counselor from said clinical supervision procedure; wherein said supervised data is used in said intervention/treatment management.

18- The digital management plan of claim 17, wherein said digital behavior management procedure, said clinical supervision and by interpreting the output data gained from each one of said digital behavior procedure and clinical supervision these procedures determine the need for a digital intervention/treatment management procedure; having two types clinical-based intervention treatment and technological-based intervention treatment.

19- The digital management plan of claim 18, wherein said clinical-based treatments comprises the following steps:
1) gathering reports from said diagnosis and behavior management and said clinical supervision performed by counselors; and
2) analyzing said gathered reports and determining an onset occurrence of a crisis for said patient; and participating in said crisis management procedure when said analyzed report indicates a crisis.

20- The digital management plan of claim 19, wherein said technological-based intervention/treatment has various treatments comprising but not limited to: psychotherapeutic mindfulness determined by said counselors; psychotherapeutic interventions determined by doctors inspecting said patient's chatbox, applications and smart clothes; gamified interventions continued by checking said patient's applications, smart clothes and extended reality; preventive interventions; psychotherapeutic CBT; supportive interventions; skills training; behavioral intervention.

21- The digital management plan of claim 20, wherein said patient's moods, symptoms and behavior data is tracked and analyzed in order to determine any indication for said crisis management procedure; wherein said mood, symptom and behavior gathered data is analyzed for further monitoring and analysis in said symptom management procedure and further reported to said report management procedure in order to prepare a comprehensive report of said patient's improvement and reaction to said treatment developed for them.

22- The digital management plan of claim 21, wherein in said digital behavior management procedure first relevant behavioral data is gathered passively via said patient's mobile/digital sensor and tracking their behavior pattern, and second relevant behavioral data is actively gathered via interactions with said patients and their said supporters in parallel; wherein said first and second relevant behavioral data is checked and combined together; wherein said combined date is cleaned and analyzed; wherein the final result gained from said clean and analyzed data is interpreted to identify and track behavioral patterns associated with each one of said patient and their said supporters, and wherein said final results and their analytic interpretation goes through detecting inconsistency procedure, report management procedure and crisis management procedure, taking proper action based on said patient's indications in said behavioral patterns

23- The digital management plan of claim 22, wherein said report management procedure comprises two approaches, first with respect to reports by said counselors and second with respect to system engines; wherein in said first report information gathered in digital diagnosis and said intake management procedures is used to scribe and draft said first reports; and wherein second information in the format of videos and audio content received from said patient during treatment process is tagged with suitable labels, then said first information is joined with said second information and said gathered behavioral patterns and changes in said mood and said symptoms in said screening and said symptom management procedures, are employed creating a full and thorough clinical reports by said counselors.

24- The digital management plan of claim 23, wherein a third relevant information is collected from all of said intake management procedure, said digital behavior management procedure, said screening management procedure, said intervention/treatment management procedure, said clinical supervision procedure, symptom management procedure, said social network management procedure and said crisis management procedure, and is given to said system report engine preparing appropriate intake report, digital behavior report, screening report, intervention/treatment report, counselor prepared report, symptom management report and social network report; wherein said aforementioned reports is used to examine effectiveness of said developed treatment plan and tracking said patient's mental state improvement.
